(19) United States
(12) Patent Application Publication
(10) Pub. No.: US 2019/0167959 A1
(43) Pub. Date: Jun. 6, 2019

(54) BIO-ARTIFICIAL PANCREAS FOR TYPE II DIABETES INTERVENTION WITH NO NEED OF IMMUNOSUPPRESSION

(71) Applicant: Taylor G. Wang, Los Angeles, CA (US)

(72) Inventor: Taylor G. Wang, Los Angeles, CA (US)

(21) Appl. No.: 15/828,860

(22) Filed: Dec. 1, 2017

(51) Int. Cl. A61M 31/00; A61L 27/26; A61L 27/56; A61L 27/04; A61F 2/02

(52) U.S. Cl. CPC A61M 31/002; A61L 27/26; A61M 2205/04; A61L 27/047; A61F 2/022; A61L 27/56

(57) ABSTRACT

A vascularized bio-artificial pancreas for managing diabetes may include a capsule having a semi-permeable interwoven capsular membrane with tapered conduits, wherein the tapered conduits are smaller in diameter proximate to an outer surface of the semi-permeable capsular membrane and larger in diameter proximate to an inner surface of the semi-permeable capsular membrane; a capsular bead encasing a plurality of the capsules; and a capsular pouch encasing the capsular bead and designed to anchor the capsular bead to a transplantation recipient.

### BACKGROUND

The embodiments described herein relate generally to diabetes management, and more particularly, to a vascularized bio-artificial pancreas for Type II diabetes interventions with no need of immunosuppression.

Encapsulation systems have been previously proposed as bio-artificial pancreases. However these designs were based on the assumption that a thin semi-permeable polymer membrane with uniform pores could keep the immune system from entering the capsules, while simultaneously allowing nutrients, oxygen, and insulin to pass in and out with ease so that the encapsulated islets would thrive and function normally in the patient. However, in actuality, polymers are random network systems whose pores are not uniform. Thus, the over-simplified assumption of membrane design could not deliver the desired transplantation results, and interest in encapsulation waned.

An encapsulation system's performance is defined by five parameters: biocompatibility, immunoprotection, mass transport, bio-stability, and mechanical strength. Because the above described design was a binary system, modification of one parameter would likely modify all other parameters, thus creating an unworkable solution.

Islet transplantations have been applied to Type I diabetes mostly. Islet transplantations with immunosuppression are not as beneficial in preventing the onset, progression and complications of Type 2 diabetes.

Therefore, what is needed is a vascularized bio-artificial pancreas for islet transplantation with no need of immunosuppressive or anti-inflammatory therapy, providing for early interventions for Type II diabetes and the potential for curing of diabetes. Specifically, what is needed is an encapsulation system to keep out all high molecular weight immune system components, such as immunoglobulin G (IgG) and immunoglobulin M (IgM), while allowing low molecular weight oxygen, nutrients, and hormones to pass.

### SUMMARY

Some embodiments of the present disclosure include a vascularized bio-artificial pancreas for managing diabetes, wherein the bio-artificial pancreas comprises three sub-systems for diabetes management. The vascularized bio-artificial pancreas may include; a capsule having an interwoven capsular membrane with tapered conduits, wherein the tapered conduits are smaller in diameter proximate to an outer surface of the semi-permeable capsular membrane and larger in diameter proximate to an inner surface of the semi-permeable capsular membrane; a capsular bead encasing a plurality of the capsules; and a capsular pouch encasing a plurality of capsular beads together to stimulate the vascular growth for mass transport. The capsule design may improve insulin release without compromising immunosuppression. The bead may ensure sufficient open space to prevent capsules from overcrowding for the health of the islets, wherein the open space may also serve as a reservoir to reduce insulin concentration fluctuations for diabetes management. The capsular pouch design may improve nutrient, oxygen, and insulin transport via vascular circulatory system. The three subsystems working together may mimic the design and function of a healthy pancreas for diabetes management, and missing any one part of the design may hasten the return of diabetes.

### BRIEF DESCRIPTION OF THE FIGURES

The detailed description of some embodiments of the invention is made below with reference to the accompanying figures, wherein like numerals represent corresponding parts of the figures.

FIG. 1A is a graph showing experimental data from a comparative example.

FIG. 1B is a graph showing experimental data from a comparative example.

FIG. 2A is a picture of a capsular bead of the present disclosure.

FIG. 2B is a picture of a capsular pouch of the present disclosure.

FIG. 3A is a graph showing experimental data from a comparative example.

FIG. 3B is a graph showing experimental data from use of an exemplary vascularized bio-artificial pancreas of the present disclosure.

FIG. 4A is a graph showing experimental data from use of an exemplary vascularized bio-artificial pancreas of the present disclosure.

FIG. 4B is a graph showing experimental data from use of an exemplary vascularized bio-artificial pancreas of the present disclosure.

FIG. 4C is a graph showing experimental data from use of an exemplary vascularized bio-artificial pancreas of the present disclosure.

FIG. 5 is a table showing experimental data from use of an exemplary vascularized bio-artificial pancreas of the present disclosure.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used as a vascularized bio-artificial pancreas for diabetes management and may comprise the following elements. This list of possible constituent elements is intended to be exemplary only, and it is not intended that this list be used to limit the encapsulated islets of the present application to just these elements. Persons having ordinary skill in the art relevant to the present disclosure may understand there to be equivalent elements that may be substituted within the present disclosure without changing the essential function or operation of the device.

1. Vascularized Bio-Artificial Pancreas

2. Capsules

3. Capsular Beads

4. Capsular Pouches

The various elements of the vascularized bio-artificial pancreas of the present disclosure may be related in the following exemplary fashion. It is not intended to limit the scope or nature of the relationships between the various elements and the following examples are presented as illustrative examples only.

By way of example, some embodiments of the present invention comprise a vascularized bio-artificial pancreas system to improve the mass transport performance without compromising immunoprotection of islet transplantation for diabetes management. The vascularized bio-artificial pancreas of the present disclosure offers encapsulated islets a separate venue for mass transport as not to compromise the immunoprotection of the capsules. The vascularized bio-artificial pancreas of the present disclosure may comprise a system with five polymers, wherein this multicomponent system may offer more flexibility in capsular design and may successfully intervene Type I and Type II diabetes without the need for immunosuppressive or anti-inflammatory therapy.

The vascularized bio-artificial pancreas of the present disclosure comprises a plurality of subsystems—specifically (i) capsules with tapered conduits, (ii) capsular beads, and (iii) capsular pouches. The vascularized bio-artificial pancreas may provide good immunoprotection and dependable and efficient mass transport. Moreover, the vascularized bio-artificial pancreas may replicate the functions of a normal, healthy pancreas.

The capsules with tapered conduits may comprise alginate (Alg)/poly L-lysine (PLL)—polymethylene-co-guanidine (PMCG)/cellulose sulfate (CS) capsules. The Alg/PLL-PMCG/CS capsules may improve immunoprotection. To improve its mass transport, capsular membrane pores may be tapered to increase transport efficiency across the membrane, as described in U.S. Pat. No. 8,673,294, the entire contents of which is herein incorporated by reference.

The capsular beads may be incorporated to prevent capsules from overcrowding and clumping to each other. Moreover, they may serve as insulin reservoirs to damp out blood glucose fluctuations.

The capsular pouch may be incorporated to anchor the capsular beads to the transplantation recipients to promote vascularization between them. They may also improve insulin transport with the vascular circulatory system.

Vascularized Bio-Artificial Pancreas Construction

A multi-loop diffusion reactor was developed to improve capsule reproducibility, as described in U.S. Pat. No. 5,462,866, the entire contents of which is hereby incorporated by reference. By balancing the gravity with buoyancy force, a capsular membrane was formed in a diffusion controlled environment. The multi-loop reactor regulated the chemical environment and reaction time, while simultaneously negating gravitational sedimentation and deformation, for all capsules' production. While other fabrication systems may be used, the multi-loop diffusion reactor may offer the most favorable and reproducible capsule fabrication.

Capsules with tapered conduits: Capsules with tapered conduits were fabricated with the following polymer combinations: Alg (about 1 to about 2%)—CS (about 0.6 to about 1.2%) and PMCG (about 0.5 to about 1.0%)—PLL (about 0.025% to about 0.05%) and CaCl₂) (about 1 to about 3%). Polyanion Alg-CS droplets were introduced into a gelling solution of CaCl₂) being continuously replenished upstream. Ca²⁺ entered the Alg-CS mixture to initiate the gelling process of CS-Ca-Alg with pores about 30 nm in diameter. Polymer beads of CS-Ca-Alg were introduced into PMCG and PLL solution after triple washes. PMCG bonded with CS preferentially and formed a PMCG-CS/Ca-Alg layer with pores about 20 nm in diameter. PLL with the largest molecular weight and lowest concentration took longer time to complete a thin and stable PMCG-CS/PLL-Alg layer over the capsules surface with pores of about 15 nm in diameter. As the membrane continued to grow inwardly, the recent completed PMCG-CS/PLL-Alg layer at the outer surface and with its small pores started to retard the rate of PMCG being replenished. The innermost PMCG-CS/Ca-Alg would thus have a lower PMCG concentration than the rest of the layer and, therefore, had larger pores with a diameter of from about 25 to about 30 nm.

The interwoven membrane layers formation started together when CS-Ca-Alg beads were introduced into PMCG and PLL solution. This protocol created a smooth transition between layers with a gradual tapered pore size distribution. This dynamic process stopped when the capsules was dumped into saline bathes to freeze in the membrane layers' structure and pore size distribution. The tapered conduits had the largest pores (about 30 nm in diameter) at inner surface of the capsule, and had smallest pores (about 15 nm) at outer surfaces. These tapered conduits were estimated to improve mass transport by a factor of three to four without compromising immunoprotection.

Capsular Beads:

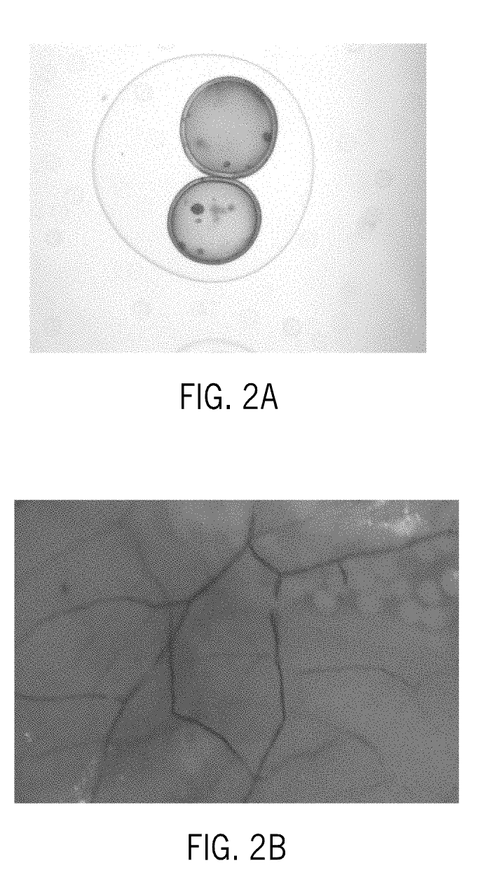

Capsules were introduced into a diluted polyanion Alg-CS solution at a volumetric ratio of 1:4. Capsular beads having a diameter of about 2 to 3 mm were produced using the same procedure as described above with respect to forming the capsules. The beads were designed to keep the capsuled from over crowding and clumping to each other. As shown in FIG. 2A, each bead contained about 2 to 4 capsules with tapered conduits, wherein the spacing is sufficient for keeping the capsules from competing with each other for nutrients and oxygen. Additionally, each capsule may contain about 5 islets. Moreover, capsular beads may serve as an insulin reservoir to smooth out blood glucose fluctuations, and provide added immune isolation protection against transplantation shock.

Capsular Pouches:

Polymeric beads were mixed with a dilute polyanion Alginate solution of equal volume to form capsular pouches for intraperitoneal (IP) and subcutaneous (SQ) transplantations. Capsular pouches were designed to anchor these capsules on IP surfaces or SQ spaces. As shown in FIG. 2B, which is a picture taken immediately after a capsule pouch was retrieved from a non-human-primate transplantation, the neovascularization network permeates throughout the capsular pouches, the capsules remain intact, and the islets remain healthy. In embodiments, IP pouches may contain about 30,000 islets each, and SQ pouches may contain about 5,000 islets each. Because the capsular pouches may be pliable, they may form intimate contact with the transplant recipient to promote micro capillary growth between the recipient's interior surface and the capsular beads.

Vascularized Bio-Artificial Pancreas Transplantation

Most Type II diabetic patients experience progressive loss of islet function, wherein the overworked islets seem to burn out, and the patients eventually need insulin injections or islet transplantations to manage their diabetes. Islet transplantations with immunosuppression are not as beneficial in preventing the onset, progression and complications of Type 2 diabetes. Thus, Type II diabetic patients may benefit the most from the vascularized bio-artificial pancreas of the present disclosure.

For Type I diabetes or Type II diabetes with end-stage renal disease, the vascularized bio-artificial pancreas may offer islet transplantation without the harmful side effects of immunosuppression or anti-inflammatory therapy. Thus, the transplantation may be easier to tolerate.

### Example

A non-human primate (NHP) model was chosen as a test subject to study the safety and efficacy of the vascularized bio-artificial pancreas (VBAP) of the present disclosure on Type II diabetes intervention. The NHP shared over 90% DNA with a human. Therefore, the VBAP transplantation experiment in the NHP model may be more valuable for future human clinical trials. However, the NHP was a difficult patient for a diabetes study, because it needed 10 times more insulin/kg than a human to manage its diabetes. Moreover, the NHP was temperamental, fragile, and did not endure stress or treatment well, which limited the number of tests, and the duration of the tests, that could be conducted.

Recipient Preparation:

NHPs (cynomolgus macaque) were rendered diabetic by injection of Streptozotocin. Blood glucose levels were monitored twice daily with blood glucose (BG) levels exceeding 300 mg/gL and c-peptide levels <0.5 ng/mL, which was considered a stable Type II diabetic state.

Islet Isolation:

NHP pancreases were surgically harvested from donor animals of either sex. Briefly, the animals were anesthetized and a surgical field created. The pancreas was partly mobilized, taking care not to sever any major blood vessels. The pancreatic duct was identified and cannulated at the duodenum. The animal was euthanized, and the excision of the pancreas was completed. NHP islets were isolated, and pancreatic islet cells were isolated from the donor pancreases.

Transplantation Protocol:

On the day of transplantation, general anesthesia was induces. For subcutaneous isle transplantation, small skin incisions were made on the antero-lateral abdominal wall, where small pockets were created in subcutaneous fat tissue, and capsular pouches were placed therein. A total of 36 capsular cylindrical in two transplantations (about 5,000 islets/cylinder) were administered into the subcutaneous fat pockets, and incisions were closed in two layers.

Upon anesthetic recovery, the animals were transferred to their cages and provided with their daily food ration.

Following islet transplantation, recipient primates were treated continuously with exogenous insulin to preserve the pre-transplantation baseline. Biopsy samples of the encapsulated islets were obtained on a monthly basis for 3 consecutive months. The samples underwent routine microscopic and immune histochemical evaluation.

Mass Transport Results

Intravenous glucose tolerance tests (IVGTTs) were performed on NHPs at 12 weeks after transplantation. Experimental and control canines received 50% dextrose (300 mg/kg) administered intravenously to all at T=0 min. Samples were collected for the determination of plasma glucose.

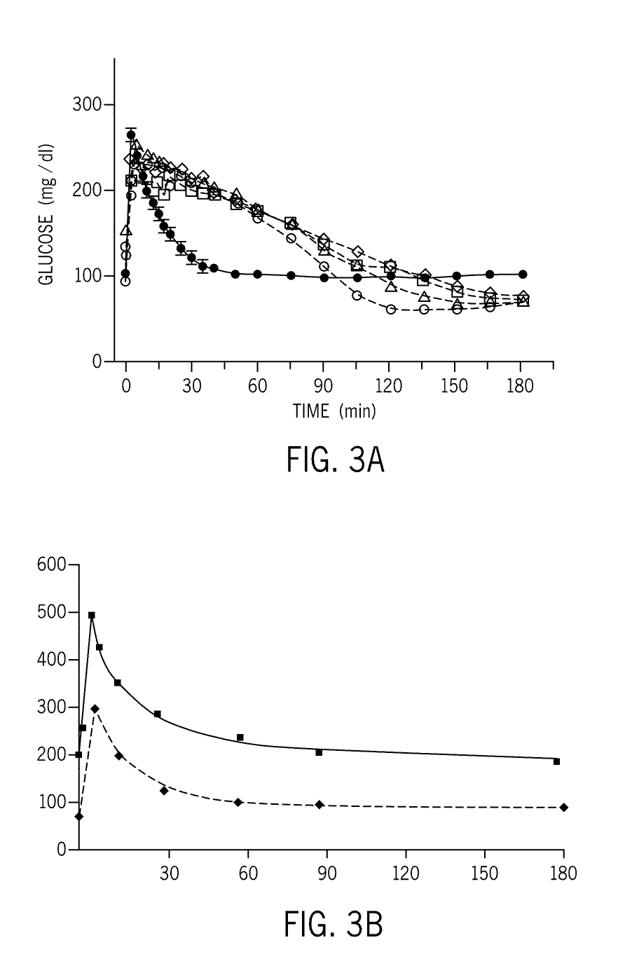

As a reference and as shown in FIG. 3A, canine encapsulation islet IVGTT data showed that blood glucose shot up quickly, returned to baseline slowly, and then overshot its baseline. The transplantation recipient suffered an extended period of hypo- and hyper-glycemic attacks, which affected the recipient's health and transplant longevity. The data shown in FIG. 3A are IVTTs from 5 controlled canines and 4 experimental canines, wherein the capsules in canines transplantation experiment had no tapered conduits.

NHP IVGTT data showed controlled animal blood glucose concentration rose from a baseline of 100 mg/dl to 300 mg/dl within 5 minutes and returned to baseline by 90 minutes. As shown in FIG. 3B, IVGTT data from an experimental animal with tapered conduits showed blood glucose concentration rose from a baseline of 200 mg/dl to 500 mg/dl within 5 minutes and returned to baseline within 95 minutes. This data was collected within 12 weeks from the capsular pouch transplant. The near identical rate of return to baseline between control and experimental animals suggests that the vascularized bio-artificial pancreas suffered minimum retardation in insulin production and secretion for diabetes management. The mass transport of the vascularized bio-artificial pancreas matched a healthy pancreas.

Type II Diabetes Intervention

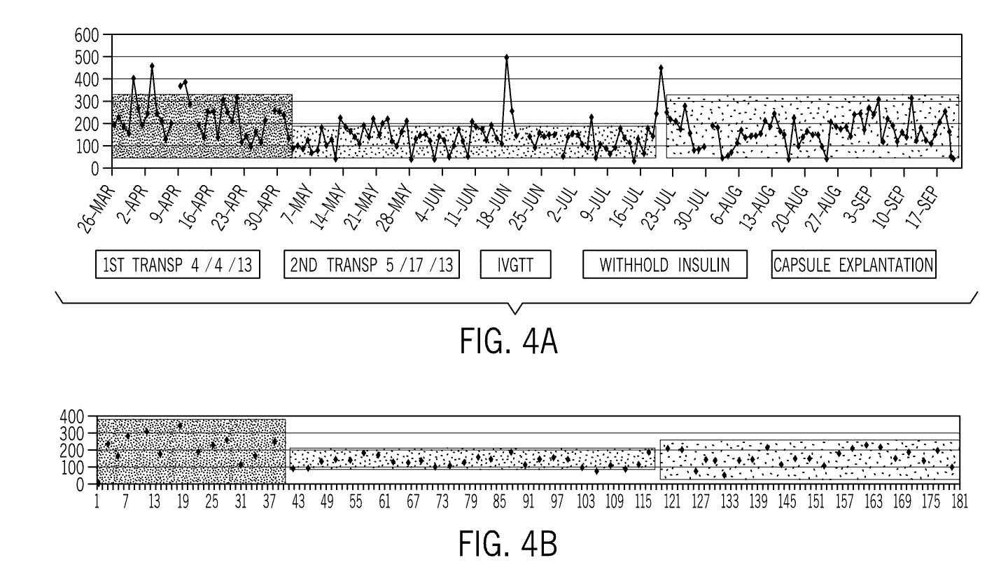

At the start of the trial, the experimental animal NHP's physiological condition was a “healthy Type II diabetic animal with its BG not under good control.” This is evidenced by the first section (the darkest section) in FIG. 4A, which shows daily blood glucose measurements. The experimental NHP (5 kg in bodyweight) received 180,000 encapsulated NHP IEQ in two transplantations. The NHP continued to receive fixed dosages of insulin therapy to preserve the pre-transplantation baseline.

The experimental animal's diabetes condition improved after six weeks of incubation period post intervention of the bio-artificial pancreas. The transplantation was well tolerated and biocompatible. About 180,000 encapsulated NHP islets (about one eighth of the therapeutic dose) was able to adjust its insulin/glucagon output to manage animal diabetes. The vascularized bio-artificial pancreas was able to manage the animal's BG level between 50 and 180 mg/dL, as shown in the middle section of FIG. 4A.

To confirm that the improvement of NHP diabetes was a direct result of the bio-artificial pancreas intervention, the encapsulated islets were explanted from the animal two months after normal glycemic levels were firmly established. Approximately 50% of the transplanted islets could be removed. The first week following the encapsulated islet explanation, the BG was lower, which was attributed to the release of stored insulin from damages islets. Soon after, the hypo- and hyper-glycemic attacks resumed and its pre-transplant ferocity, if not more, as shown in the last section of FIG. 4A.

FIG. 4B shows three day average blood glucose measurements of FIG. 4A. FIG. 4C shows the daily insulin supplements, wherein the regular insulin is the lighter shading and the long acting insulin is the darker shading.

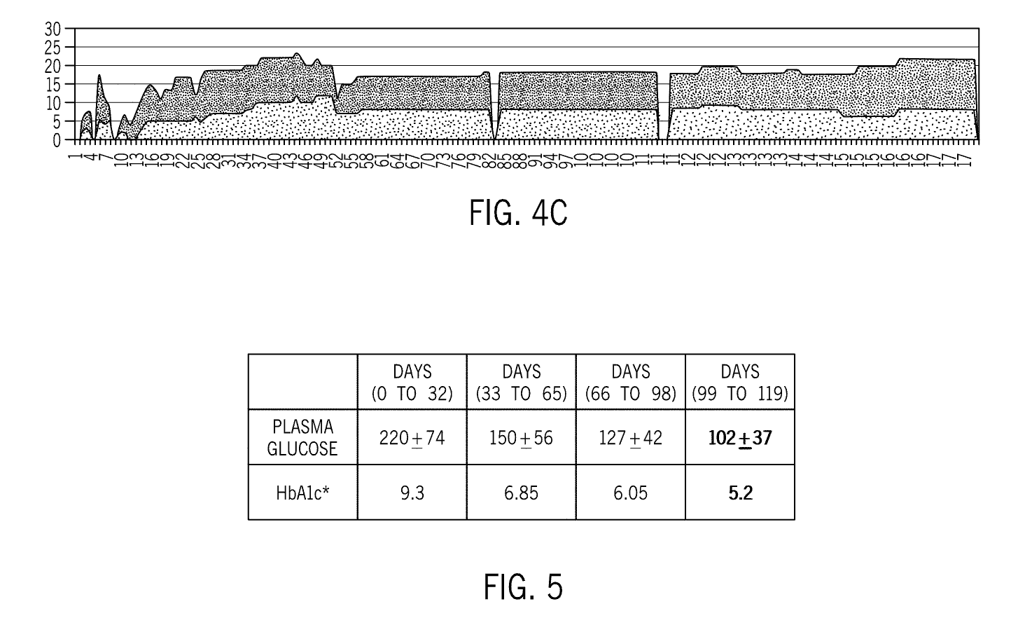

As shown in the table in FIG. 5, the NHP with the encapsulated islets showed steady improvement in diabetic management, which suggested that the vascularized bio-artificial pancreas was functioning as designed. The transplant recipient was healthier, and encapsulated islets were regulating the BG in the Type II diabetic NHP. The table shows the post-transplantation fasting plasma glucose measurements, wherein the date shows that the vascularized bio-artificial pancreas has the potential for curing diabetes. The HbA1c row shows values that were derived from average plasma glucose numbers for comparison purposes.

### Comparative Example

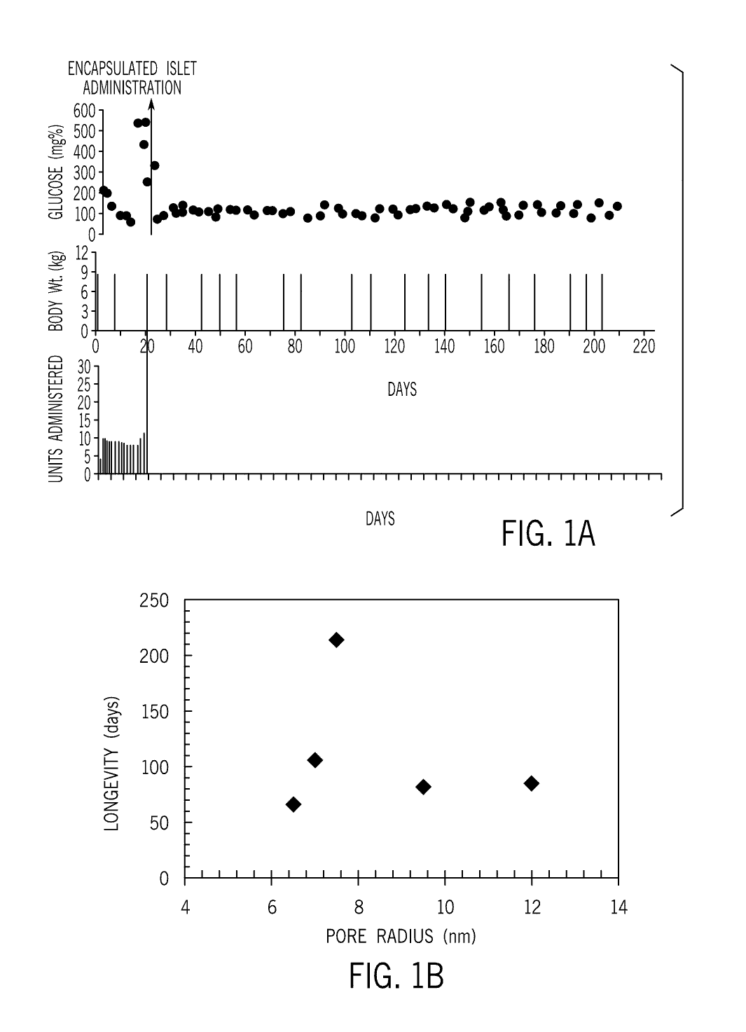

As compared to the vascularized bio-artificial pancreas of the present disclosure, a bio-artificial model with a thicker membrane with non-tapered pore size distribution (˜15 nm in diameter) was chosen for an immunoprotection study in a large animal model (canine). Encapsulated islets worked well in canine allo-transplantations, wherein fasting blood glucose levels were normalized in nine out of nine total pancreatectomized dogs for up to 214 days with a single transplantation, as shown in FIG. 1A. No immunosuppressive or anti-inflammatory therapy was used or needed.

Specifically, with respect to FIG. 1A, a canine weighing 8.3 kg received 85,000 IEQ/kg encapsulated islets intraperitoneally and maintained normal glycemia for 214 days. The top panel of FIG. 1A displays data points that correspond to venous plasma glucose concentrations collected following a meal. The middle panel of FIG. 1A displays vertical bars representing the body weight of the animal. The bottom panel of FIG. 1A displays exogenous insulin administered prior to transplantation. The experiment was terminated when encapsulated islets failed to maintain a fasting glycemia of less than 180 mg/dl for three consecutive days.

However, over time (<200 days), the animal's general health started to decline. Specifically, experimental animal circulating plasma insulin concentrations started to decrease. Canine intravenous glucose tolerance test (IVGTT)/oral glucose tolerance test (OGTT) blood glucose show up higher and returned to baseline slower and then overshot its baseline. Transplantation recipients suffered extended periods of hyperglycemia and hypoglycemia episodes as a consequence of diminishing and delayed insulin release.

Aside from elevated fasting glycemic levels, the animal's general appearance was lethargic. This extended period of hyperglycemia and hypoglycemia hastened the return of diabetes in less than 3/5 of a year in canine experiments. Capsules retrieved from experimental animals were free-floating, and their surface was free of fibrosis. On the other hand, retrieved islet cells ranged from a normal appearance to exhibiting shrunken, pyknotic nuclei, loss of cellular margin, and mineralization.

This suggested failure of the transplant was not likely caused by biocompatibility or immunoprotection failure, but rather by insufficient insulin production and release, which suggests that increased capsular membrane thickness improved immunoprotection at the expense of insulin mass transport. This resulted in the gradual deterioration of the recipient's health. Efforts to improve transplant longevity by adjusting its membrane parameters were met with limited success. If the pores were increased in size to improve mass transport, the immunoprotection would be compromised; and if the membrane was increased in thickness to improve immunoprotection, the mass transport would be marginalized. Thus, as shown in FIG. 1B, the dichotomous limitations between immunoprotection and mass transport placed unacceptable limits on transplant longevity.

With regard to FIG. 1B, a total of eight canines received encapsulated islets of different membrane pore sizes intraperitoneally and maintained exogenous insulin independence with a single transplantation. Longevity data for 9 nm pore radius was the average of three transplantations, and longevity data for 12 nm pore radios was the average of two transplantations.

Therefore, transplant longevity was capped by the compromise between immunoprotection and mass transport designs.

### CONCLUSION

To prevent Type II diabetes' progression and complications, the vascularized bio-artificial pancreas of the present disclosure may offer a sub-therapeutic dosage of encapsulated islets' transplantation to take over some of the load of insulin production from burned out islets, allowing them a chance to rest, recover, and, if necessary, replace, thus arresting the progression of Type II diabetes.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A vascularized bio-artificial pancreas for managing diabetes, the vascularized bio-artificial pancreas comprising:
a capsule having a semi-permeable interwoven capsular membrane with tapered conduits, wherein the tapered conduits are smaller in diameter proximate to an outer surface of the semi-permeable capsular membrane and larger in diameter proximate to an inner surface of the semi-permeable capsular membrane;
a capsular bead encasing a plurality of the capsules; and
a capsular pouch encasing the capsular bead and designed to anchor the capsular bead to a transplantation recipient.

2. The vascularized bio-artificial pancreas of claim 1, wherein the semi-permeable capsular membrane comprises a plurality of membrane layers.

3. The vascularized bio-artificial pancreas of claim 2, wherein comprises the following membrane layers in order from the outer surface to the inner surface:
a polymethylene-co-guanidine (PMCG)-cellulose sulfate (CS)/poly L-lysine (PLL)-Alginate (Alg) layer; and
a PMCG-CS/Ca-Alg layer.

4. The vascularized bio-artificial pancreas of claim 3, wherein:
the PMCG-CS/PLL-Alg layer comprises pores having a diameter of about 15 nm; and
the PMCG-CS/CA-Alg layer comprises larger pores with a diameter of from about 20 to about 30 nm.

5. The vascularized bio-artificial pancreas of claim 3, wherein the capsules comprise:
about 1 to about 2% alginate;
about 0.6 to about 1.2% cellulose sulfate;
about 0.5 to about 1.0% PMCG;
about 0.025 to about 0.05% PLL; and
about 1 to about 3% CaCl₂).

6. The vascularized bio-artificial pancreas of claim 1, wherein the capsular bead has a diameter of about 2 to 3 mm.

7. The vascularized bio-artificial pancreas of claim 1, wherein the capsular bead contains about 2 to about 4 capsules encased therein.

8. The vascularized bio-artificial pancreas of claim 1, wherein the vascularized bio-artificial pancreas comprises a plurality of capsular pouches, each capsular pouch encasing a plurality of capsular beads:
about 30,000 islets for intraperitoneal transplantations; and
about 5,000 islets for subcutaneous transplantations.

9. The vascularized bio-artificial pancreas of claim 1, wherein the capsular bead comprises:
about 0.5 to about 1% alginate;
about 0.3 to about 0.6% cellulose sulfate; and
about 1 to about 3% CaCl₂).

10. The vascularized bio-artificial pancreas of claim 1, wherein the capsular pouch comprises:
about 0.5 to about 1% alginate; and
about 1 to about 3% CaCl₂).
